(19) United States
(12) Patent Application Publication
(10) Pub. No.: US 2017/0183691 A1
(43) Pub. Date: Jun. 29, 2017

(54) SIMULTANEOUS SACCHARIFICATION AND CO-FERMENTATION OF GLUCOAMYLASE-EXPRESSING FUNGAL STRAINS WITH AN ETHANOLOGEN TO PRODUCE ALCOHOL FROM CORN

(71) Applicant: Danisco US Inc., Palo Alto, CA (US)

(72) Inventors: Kathleen A. Clarkson, San Francisco, CA (US); Matthew T. Reboli, Sunnyvale, CA (US); Jacquelyn A. Huitink, Burlingame, CA (US); Paula Johanna Maria Teunissen, Saratoga, CA (US); Gopal K. Chotani, Cupertino, CA (US); Jayarama K. Shetty, Pleasanton, CA (US)

(73) Assignee: Danisco US Inc., Palo Alto, CA (US)

(21) Appl. No.: 15/305,036

(22) PCT Filed: Apr. 6, 2015
(86) PCT No.: PCT/US2015/024522

§ 371 Date: Oct. 18, 2016

Related U.S. Application Data
(60) Provisional application No. 61/982,199, filed on Apr. 21, 2014.

(51) Int. Cl. C12P 7/14

(52) U.S. Cl. CPC C12P 7/14

(57) ABSTRACT

A conversion process provides using different co-cultured cell lines to express different sets of enzymes catalyzing the same process. For example, in a Simultaneous Saccharification and Co-Fermentation (SSCF) process, a starch substrate is converted to alcohol by contacting the substrate with yeast and Aspergillus niger cells. Because A. niger expresses an endogenous glucoamylase and alpha-amylase, these enzymes do not need to be added during the SSCF process.

### CROSS REFERENCE TO RELATED APPLICATIONS

This application claims benefit of priority from US provisional application U.S. Ser. No. 61/982,199, filed 21 Apr. 2014 and is incorporated herein by reference in their entirety.

### BACKGROUND

Bioconversion of biomass has significant advantages over other alternative energy strategies because biomass is both abundant and renewable. Bioconversion can be performed by co-culturing two or more fungal strains in mixed culture fermentation. Mixed fungal cultures have many advantages compared to their monocultures, including improving productivity, adaptability, and substrate utilization. (Dashtban et al., Int. J. Biol. Sci., 5:578-595, 2009.) Co-establishment of a stable co-culture has been reported to depend on media and growth requirements, such as temperature, atmosphere and carbon source. (Maki et al., Int. J. Biol. Sci., 5:500-516, 2009.) Co-cultures have been reported to be affected by metabolic interactions (e.g., syntrophic relationships or alternatively competition for substrates) and other interactions (e.g., growth promoting or growth inhibiting such as antibiotics). (See, e.g., Maki et al., Int. J. Biol. Sci., 5:500-516, 2009.)

Solid state co-fermentation (e.g., using fermentation trays) of two fungal strains has been reported (see, e.g., Sun et al., Electronic J. Biotechnol., 12: 1-13, 2008; Pandey et al., Curr. Sci., 77:149-162, 1999; Hu et al., Int'l Biodeterioration & Biodegradation 65:248-252, 2011; Wang et al., Appl. Microbiol. Biotechnol. 73:533-540, 2006). However, solid state co-fermentations are difficult, cost prohibitive for industrial applications, and thus not always suitable for recombinant production of enzymes at industrial scales. Submerged fermentations are often more flexible and deemed more desirable, which have been used on, for example, Penicillium sp. CH-TE-001 and Aspergillus terreus CH-TE-013 for producing an enzyme mixture (Garcia-Kirchner, et al., Applied Biochem. & Biotechnol. 98:1105-1114, 2002). In addition, mixed cultures of microorganisms have been fermented under different conditions to obtain cultivated microorganisms enriched for certain characteristics, which are then blended to obtain a formulated complex culture (see, e.g., EP 2292731).

The primary method for production of fuel ethanol involves the hydrolysis of starch or grain into glucose followed by a yeast fermentation to the final product, ethanol. Typically, the hydrolysis of corn starch into glucose and fermentation into ethanol occurs simultaneously in a process commonly referred to as Simultaneous Saccharification and Fermentation (SSF). Corn starch must undergo several processes before yeast can ferment the glucose to ethanol. Throughout the corn starch cooking process, starch is exposed to several types of enzymes to catalyze the conversion of long chain starch molecules into smaller, fermentable sugars. Alpha-amylases, in combination with high temperature, catalyze the random hydrolysis of starch enabling liquefaction in preparation for SSF. During SSF, glucoamylases and additional alpha-amylases, which act together in a synergistic reaction, hydrolyze the solubilized starch chains to fermentable sugars, such as maltose (DP2) and glucose (DP1). Products such as DISTILLASE™ SSF, DISTILLASE™ SSF+, and G-Zyme® 480 Ethanol (DuPont Industrial Biosciences), an optimized blend of an Aspergillus glucoamylase, a Bacillus licheniformis pullulanase, and a Trichoderma protease are commonly added to the fermentation to catalyze the hydrolysis of starch to glucose. Under conventional SSF processes, enzymes are added exogenously.

### BRIEF DESCRIPTION OF THE FIGURES

The accompanying drawings are incorporated in and constitute a part of this specification and illustrate various methods and compositions disclosed herein. In the drawings:

FIG. 1 depicts DP4+ hydrolysis after a 9 hour seed incubation of A. niger (blend 1) versus T. reesei (blend 3) under conventional fermentation conditions at 32° C.

FIG. 2 depicts ethanol production (% v/v) after a 9 hour seed incubation of A. niger (blend 1) versus T. reesei (blend 3) under conventional fermentation conditions at 32° C.

FIG. 3 depicts fermentation temperature profiles used in the Example.

FIG. 4 depicts final DP1 yields of control blends at each experimental temperature condition.

FIG. 5 depicts DP4+ levels at SSF time=0 for each experimental temperature condition.

FIG. 6 depicts DP1 production after a 9 hour seed incubation of A. niger (blend 1) versus T. reesei (blend 3) under conventional fermentation conditions at 32° C.

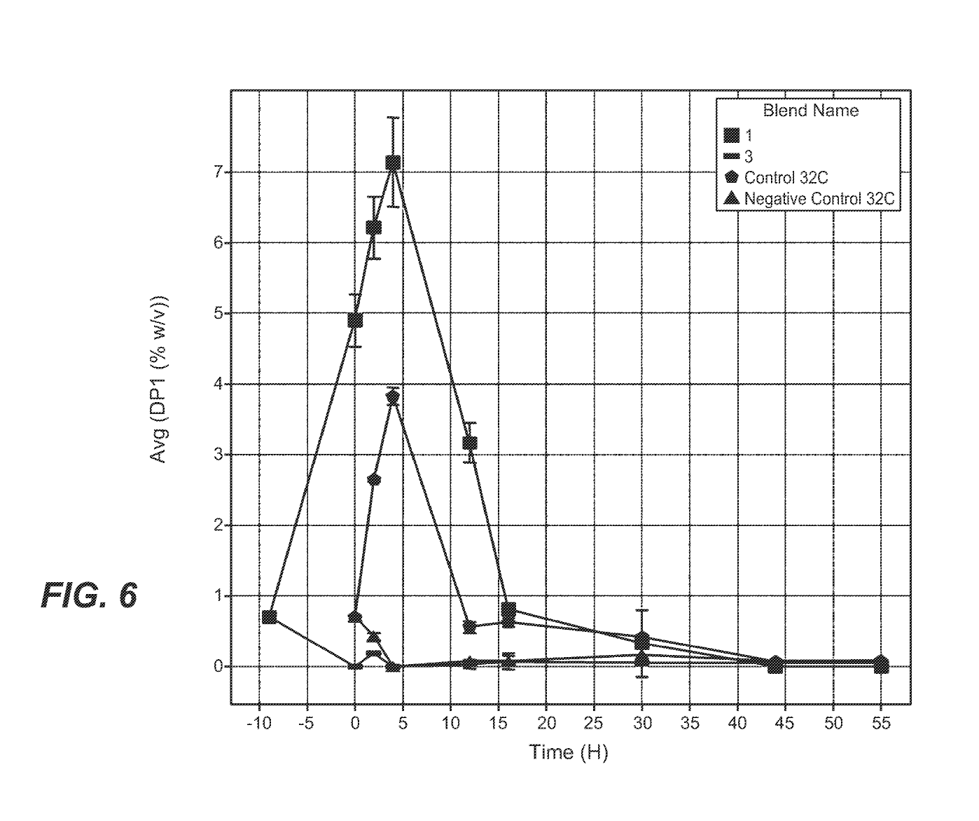

FIG. 7 depicts DP1 production after a 9 hour seed incubation of A. niger (blend 5) versus T. reesei (blend 7) under conventional fermentation conditions at 35° C.

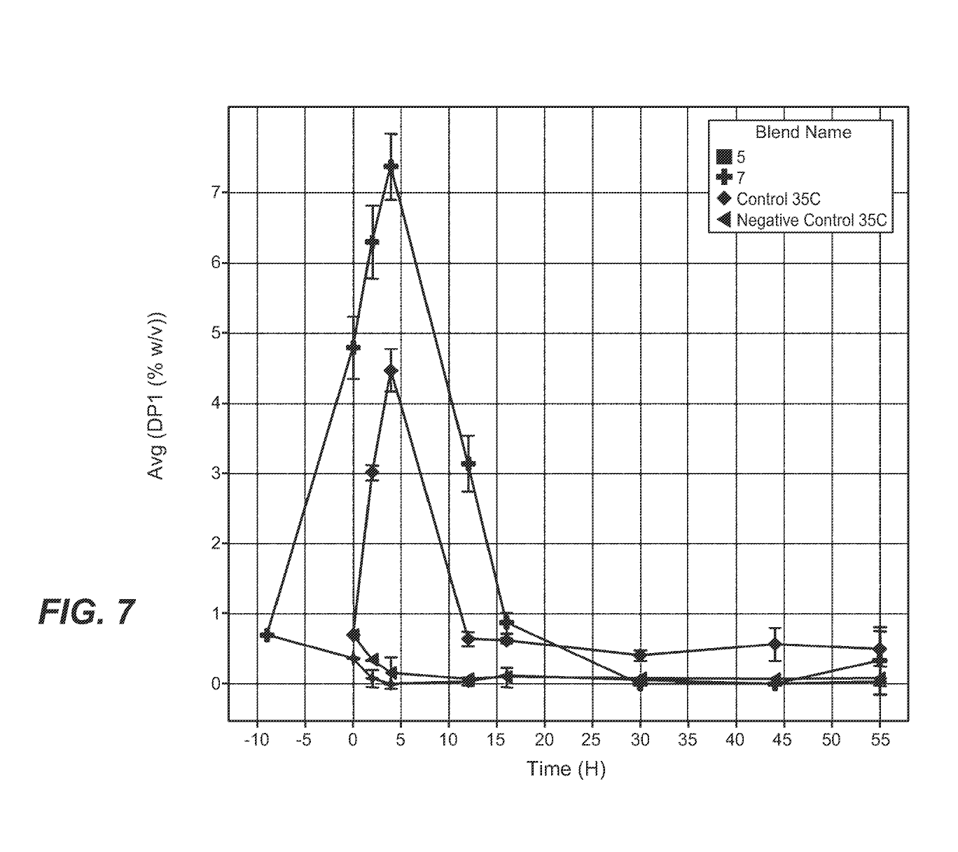

FIG. 8 depicts DP1 production after a 9 hour seed incubation of A. niger (blend 10) versus T. reesei (blend 12) under conventional fermentation conditions at a 32° C. to 38° C. staged temperature profile.

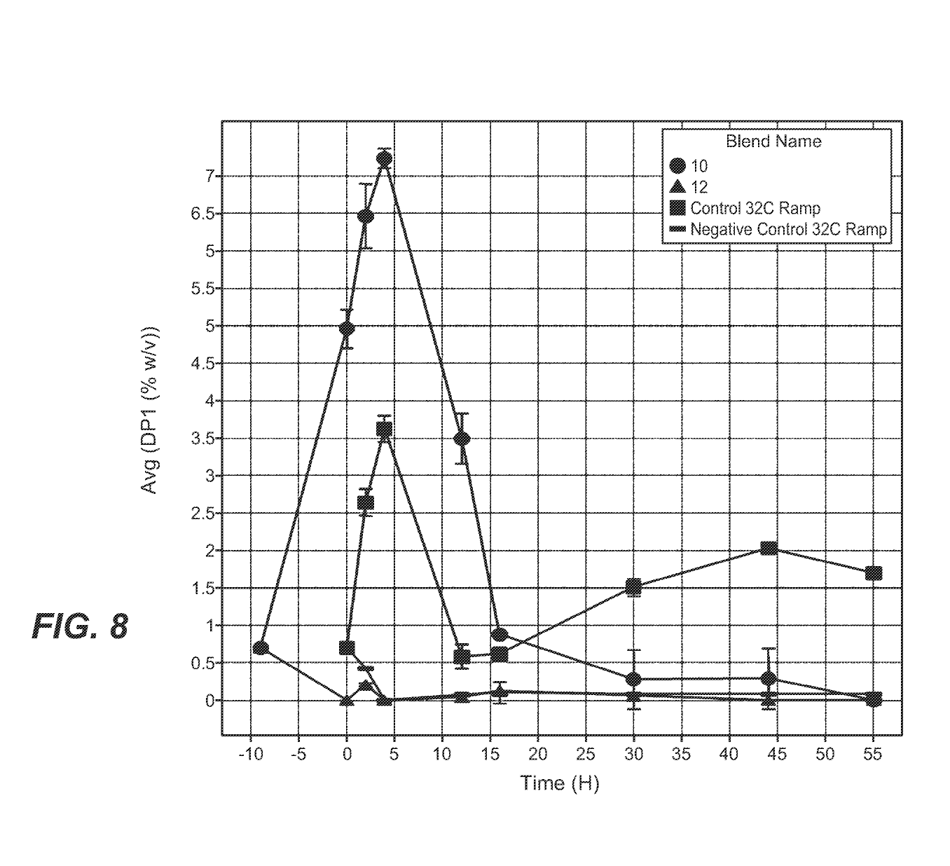

### SUMMARY

A conversion process of converting a starch substrate into a product is provided. The conversion process may be a process of converting a starch substrate by Simultaneous Saccharification and Co-Fermentation (SSCF). The conversion process comprises contacting a starch substrate with a first cell from a fungus, e.g., a yeast cell, and a second cell from a filamentous fungus, e.g., a Trichoderma or Aspergillus cell, at a temperature of about 32° C. to about 38° C. The process may comprise a pre-incubation of the second cell with the starch substrate before the second cell and starch substrate are contacted with the first cell. The starch substrate may be a liquefact or un-gelatinized starch.

The second cell may be a filamentous fungus, e.g., an A. niger cell, capable of expressing both an endogenous glucoamylase and an acid stable alpha-amylase. The conversion process may be conducted without the addition of an exogenous glucoamylase, alpha-amylase, or protease. For example, the conversion process may be conducted without each of an exogenous glucoamylase, alpha-amylase, or protease. The product may be ethanol, and the ethanol yield may be about 13% to about 14% v/v ethanol at the completion of the conversion process. Where the second cell is a T. reesei cell, the conversion process may be conducted without the addition of an exogenous glucoamylase and/or with a reduced level of other additives. In this case, the ethanol yield may be about 4% to about 8% v/v ethanol at the completion of the conversion process. The first cell is a distinct species from the second cell. For example, the yeast first cell would not be an A. niger cell, if the second cell were an A. niger cell.

A conversion process of converting a starch substrate into a product may comprise contacting a starch substrate with a yeast first cell and an Aspergillus niger second cell, wherein said conversion process produces, or is capable of producing, an alcohol yield of at least 90%, e.g., at least 93%, 95%, 97%, 98%, or 99%, over a temperature range of about 32° C. to about 38° C. at the completion of the conversion process, compared to the alcohol yield at the completion of a control process performed under comparable conditions, wherein the control process comprises contacting the starch substrate with the yeast first cell and adding an exogenous glucoamylase, fungal alpha-amylase, and optionally fungal protease and/or other enzymes, and wherein the conversion process produces the product.

The product may be alcohol, for example ethanol or butanol. The product of the conversion process may be an organic acid, e.g., citric acid, lactic acid, succinic acid, itaconic acid, levulinic acid, monosodium glutamate, a gluconate, or an amino acid, e.g., lysine tryptophan, or threonine.

The conversion process may produce an alcohol yield of at least of 95%-99%, e.g., 97%-99% or 95%-98%, at the completion of the conversion process, compared to the alcohol yield at the completion of the control process. The conversion process may be performed over a temperature range of about 32° C. to about 38° C., e.g., about 34° C., 35° C. or 36° C. to about 38° C., and may produce an alcohol yield of at least 90%%, e.g., at least 93%, 95%, 97%, 98%, or 99%, at the completion of the process, compared to the alcohol yield of the control process performed under comparable conditions. The conversion process may be performed at a temperature of about 35° C. and may produce an alcohol yield of at least 90% at the completion of the process, compared to the alcohol yield of the control process. The alcohol may be ethanol or butanol, for example.

The conversion process may comprise pre-incubating the second cell with the starch substrate before the second cell and starch substrate are contacted with the first cell. The pre-incubating may be conducted for 6-12 hours, e.g., 8-10 hours, or about 9 hours. The starch substrate may be a liquefact or granular starch. The conversion process may be conducted without the addition of an exogenous glucoamylase, non-starch hydrolyzing enzyme, alpha-amylase, phytase, and/or protease. For example, the conversion process may be conducted without the addition of some or all of the exogenous enzymes above.

The yeast first cell may express an exogenous and/or endogenous glucoamylase, non-starch hydrolyzing enzyme, alpha-amylase, phytase, and/or protease during the conversion process. For example, the yeast first cell may express an alpha-amylase from Aspergillus.

### Definitions

A control process is conducted under “comparable conditions” as the conversion process. For example, if the conversion process were conducted over a temperature range of 32-38° C., the control process would be conducted over the same temperature range, using the same temperature profile of the conversion process. The difference between the conversion process and control process is thus the presence of an Aspergillus niger second cell in the conversion process and the addition of an exogenous glucoamylase, fungal alpha-amylase, and fungal protease in the control process. Table I shows representative reaction parameters for conversion and control processes. The process is “complete” when no more product is formed.

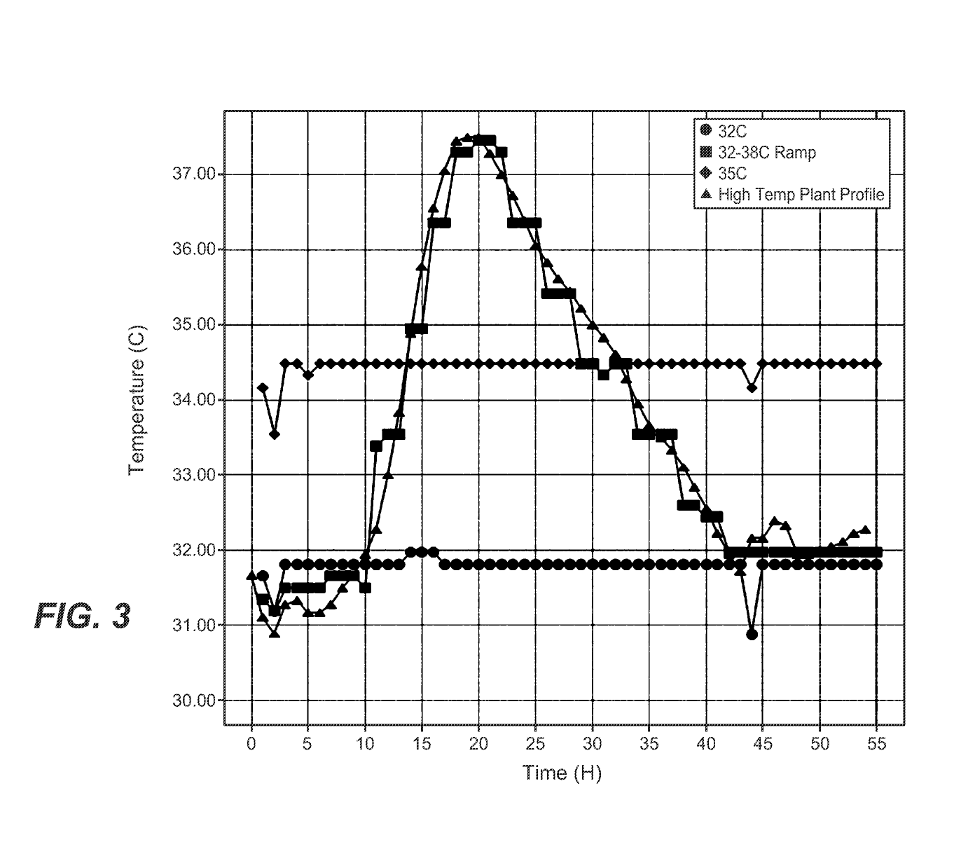

“About” refers to an average temperature during the process. The skilled artisan would expect the temperature of a conversion process to vary somewhat about a set temperature, e.g., by ±1° C. from the set value, such as depicted in FIG. 3. A temperature of “about 32° C.” thus would encompass temperatures of 32±1° C. during the conversion process. A temperature of “about 38° C.” encompasses temperatures of 38±1° C. and also includes transient spikes in temperature that can occur during the conversion process. For example, the temperature of a conversion process may exceed 38° C. by several degrees over several minutes. These transient spikes are encompassed by “about 38° C.”

The cells used in the present methods can be from any type of organism, e.g., eukaryotic organisms, prokaryotic organisms and archaebacteria. Preferably the cells are from a microorganism (i.e., microbial cell lines), meaning the cells are prokaryotic, archaebacteria, or from a eukaryote capable of unicellular growth, such as fungi (e.g., filamentous fungi or yeasts), and algae. Different organisms can be classified by domain (e.g., eukaryotes and prokaryotes). Domains are subdivided into kingdoms, e.g., Bacteria (e. g., Eubacteria); Archaebacteria; Protista; Fungi; Plantae; and Animalia. Kingdoms are further divided into phylums, classes, subclasses, orders, families, and genera. For example, genera from fungi include Trichoderma, Aspergillus, Dermatophytes, Fusarium, Penicillum, and Saccharomyces. Genera are further divided into species. For example, species from Trichoderma include Trichoderma reesei, Trichoderma viride, Trichoderma harzianum, and Trichoderma koningii. Species are divided into strains.

“Pitching” means adding a fungal strain, e.g., yeast, to a fermentation.

Different strains are independent isolates of the same species. Different strains have different genotypes and/or phenotypes.

A cell line is used in the conventional sense to indicate a population of substantially isogenic cells capable of continuous (preferably indefinite) growth and division in vitro without change other than occasional random mutations inherent from DNA replication. A cell line is typically propagated from a single colony.

Submerged fermentation is a process in which the cells grow at least predominantly under the surface of the liquid medium.

Solid state fermentation is a process in which cells grow on and inside a solid medium.

An “exogenous enzyme” means an enzyme that is not normally expressed by a cell (e.g., a heterologous enzyme from another strain, species, genera or kingdom or a recombinantly modified variant of an enzyme normally expressed by the cell) or an enzyme that is normally expressed by the cell but is expressed at an increased level by virtue of being under the control of genetic material not normally present in the cell. Such expression can result from introduction of a gene encoding such an enzyme at a location where it is not normally present or by genetic manipulation of the cell to enhance the expression of an enzyme. Such genetic manipulation can change a regulatory element controlling expression of the enzyme or can introduce genetic material encoding a protein that acts in trans to enhance expression of the enzyme.

A conversion process conducted with the “addition of an exogenous enzyme” means that an enzyme is added to the conversion reaction from an external source; i.e., a solution of an enzyme exogenous to the conversion reaction is added to the conversion reaction. While the enzyme is added exogenously to the conversion reaction, the enzyme itself does not necessarily have to be an “exogenous enzyme” in the sense used above. For example, a first cell may express a glucoamylase in a conversion process, and the same glucoamylase may be added exogenously to the same conversion process.

An exogenous nucleic acid (e.g., DNA) means a nucleic acid not normally present in a cell (i.e., introduced by genetic engineering). An exogenous nucleic acid can be from a different strain, species, genera or kingdom (i.e., heterologous), can encode recombinantly engineered variants, or can be normally present in a cell but introduced in a different location than normally present.

An enzyme is “endogenous” to a cell if the enzyme is normally expressed by the cell, and neither nucleic acid encoding the enzyme or any other nucleic acid regulating expression of the enzyme has been introduced into cell. An endogenous gene means a gene normally present in a cell at its normal genomic location. An enzyme or nucleic acid encoding the enzyme are heterologous to a cell, for example, if they are not normally encoded by the cell and introduced into the cell by genetic engineering. For example, an enzyme or nucleic acid encoding the enzyme are heterologous to the cell if an endogenous nucleic acid has been modified/engineered and/or if an endogenous unmodified or modified nucleic acid have inserted in a different location in the cell.

The term “filamentous fungi” refers to all filamentous forms of the subdivision Eumycotina (see Alexopoulos (1962) INTRODUCTORY MYCOLOGY, Wiley, New York). These fungi are characterized by a vegetative mycelium with a cell wall composed of chitin, cellulose, and other complex polysaccharides. The filamentous fungi are morphologically, physiologically, and genetically distinct from yeasts. Vegetative growth by filamentous fungi is by hyphal elongation and carbon catabolism is obligatory aerobic.

A cell is disposed to express an enzyme if the cell includes DNA encoding the enzyme operably linked to one or more regulatory elements that allow expression of the DNA. The enzyme can be endogenous or exogenous. Expression can be constitutive or inducible. The DNA encoding the enzyme can be in a genomic or episomal location within the cell. When two enzymes are said to be expressed at different levels by different cell lines, the ranges represented by the standard error of the mean (SEM) for the respective expression levels at the protein level do not overlap. Expression levels are compared between respective cultures of the same density and stage of culture growth of the respective cell lines. When the expressed protein is secreted, the expression levels are preferably determined from the concentration of secreted protein in culture media. Expression levels can be determined in units of moles, activity units, OD, or other units.

The term “about” when used to modify a parameter means that the units defining the parameter may vary ±10% from the disclosed value.

The term “butanol” as used herein refers to the butanol isomers 1-butanol (1-BuOH), 2-butanol (2-BuOH), tert-butanol (t-BuOH), and/or isobutanol (iBuOH or i-BuOH, also known as 2-methyl-1-propanol), either individually or as mixtures thereof. From time to time, as used herein the terms “biobutanol” and “bio-produced butanol” may be used synonymously with “butanol.”

In certain embodiments, the microorganism may be genetically modified to produce butanol. The production of butanol by a microorganism, is disclosed, for example, in U.S. Pat. Nos. 7,851,188; 7,993,889; 8,178,328; and 8,206,970; and U.S. Patent Application Publication Nos. 2007/0292927; 2008/0182308; 2008/0274525; 2009/0305363; 2009/0305370; 2011/0250610; 2011/0313206; 2011/0111472; 2012/0258873; and 2013/0071898, the entire contents of each are herein incorporated by reference. In certain embodiments, the microorganism is genetically modified to comprise a butanol biosynthetic pathway or a biosynthetic pathway for a butanol isomer, such as 1-butanol, 2-butanol, or isobutanol. In certain embodiments, at least one, at least two, at least three, at least four, or at least five polypeptides catalyzing substrate to product conversions in the butanol biosynthetic pathway are encoded by heterologous polynucleotides in the microorganism. In certain embodiments, all the polypeptides catalyzing substrate to product conversions of the butanol biosynthetic pathway are encoded by heterologous polynucleotides in the microorganism. In will be appreciated that microorganisms comprising a butanol biosynthetic pathway may further comprise one or more additional genetic modifications as disclosed in U.S. Patent Application Publication No. 2013/0071898, which is herein incorporated by reference in its entirety.

Biosynthetic pathways for the production of isobutanol that may be used include those as described by Donaldson et al. in U.S. Pat. No. 7,851,188; U.S. Pat. No. 7,993,388; and International Publication No. WO 2007/050671, which are incorporated herein by reference. Biosynthetic pathways for the production of 1-butanol that may be used include those described in U.S. Patent Application Publication No. 2008/0182308 and WO2007/041269, which are incorporated herein by reference. Biosynthetic pathways for the production of 2-butanol that may be used include those described by Donaldson et al. in U.S. Pat. No. 8,206,970; U.S. Patent Application Publication Nos. 2007/0292927 and 2009/0155870; International Publication Nos. WO 2007/130518 and WO 2007/130521, all of which are incorporated herein by reference.

The following abbreviations are used:

AA Alpha-amylase

ADY Active Dry Yeast

AFP Acid fungal protease

AkAA Aspergillus kawachii alpha-amylase

AnGA Aspergillus niger glucoamylase

AsAA Acid stable alpha-amylase

C Celsius

DE Dextrose equivalent

DP Degree of glucose polymerization

DS Dry solids

EoF End of Fermentation

g grams

GA Glucoamylase

GAU Glucoamylase Unit

HPLC High Performance Liquid Chromatography

mL/μL milliliter/microliter

N Normal

ppm Parts per million

rpm Revolutions per minute

SSCF Simultaneous saccharification and co-fermentation

SSF Simultaneous saccharification and fermentation

SSU Starch Saccharifying Unit

TrGA Trichoderma reesei glucoamylase

v/v volume to volume

w/v weight to volume

wt wildtype

### DETAILED DESCRIPTION

### I. Introduction

The invention provides a conversion process using different cell lines that are co-cultured. The cell lines express different sets of enzymes to catalyze the same process under a single set of defined conditions. Co-culturing provides greater flexibility and simplicity, less waste, lower energy and water utilization, and lower costs than conventional methods. It allows various enzymes mixtures to be made as needed without having to build a new production strain for each individual type of substrates and pretreatment methods. It also allows the desired enzyme mixtures to be created in one batch, obviating the need for blending the output from several separate fermentations. Preparation of a mixture of enzymes according to the present methods does not require a full recovery process for each fermentation, and/or separate storage of each enzyme component. Further, it allows maintenance of each production strain separately thereby preventing loss of the entire cocktail (engineered into a single production cell line) all at once.

### II. Conversion Process

A conversion process is a process in which a substrate is converted into a product by two or more enzymes. The substrate can be a complex substance such as plant material containing multiple types of molecules. The product can be a single product or multiple products. The conversion process can be a single step process or involves multiple steps. The process can involve multiple sequential and/or parallel steps. Different enzymes can act in sequential steps, parallel steps or in combination on the same step. Exemplary conversion processes include the conversion of cellulosic biomass, glycogen, starch and various forms thereof into sugars (e.g., glucose, xylose, maltose) and/or alcohols (e.g., methanol, ethanol, propanol, butanol).

Some conversion processes convert starch, e.g., corn starch, wheat starch, or barley starch, corn solids, wheat solids, and starches from grains and tubers (e.g., sweet potato, potato, rice and cassava starch) into ethanol, or a syrup rich in saccharides useful for fermentation, particularly maltotriose, glucose, and/or maltose, or simply into one or more forms of sugars, which are in themselves useful products.

Some conversion processes act on cellulosic or lignocellulosic material such as materials comprising cellulose and/or hemicellulose, and sometimes lignin, starch, oligosaccharides, and/or monosaccharides. Cellulosic or lignocellulosic material can optionally further comprise additional components, such as proteins and/or lipids. Cellulosic or lignocellulosic material includes bioenergy crops, agricultural residues, municipal solid waste, industrial solid waste, sludge from paper manufacture, yard waste, wood and forestry waste, such as corn cobs, crop residues such as corn husks, corn stover, grasses, wheat, wheat straw, barley straw, hay, rice straw, switchgrass, waste paper, sugar cane bagasse, sorghum, giant reed, elephant grass, miscanthus, Japanese cedar, components obtained from milling of grains, tress, branches, roots, leaves, wood chips, sawdust, shrubs and bushes, vegetables, fruits, flowers and animal manure. Cellulosic or lignocellulosic material can be derived from a single source, or can comprise a mixture derived from more than one source. For example, cellulosic or lignocellulosic material can comprise a mixture of corn cobs and corn stover, or a mixture of grass and leaves. Exemplary products of enzymatic conversion of the cellulosic or lignocellulosic material substrate are glucose and ethanol.

In other conversion processes, the substrate is glucose, fructose, dextrose, and sucrose, and/or C5 sugars such as xylose and arabinose, and mixtures thereof. Sucrose can be derived from sources such as sugar cane, sugar beets, cassava, sweet sorghum, and mixtures thereof. Glucose and dextrose can be derived from renewable grain sources through saccharification of starch based feedstocks including grains such as corn, wheat, rye, barley, oats, and mixtures thereof. Fermentable sugars can also be derived from cellulosic or lignocellulosic biomass through processes of pretreatment and saccharification. The product of such conversion processes can be alcohols such as ethanol or butanol.

In some conversion processes, the substrates are pretreated. Pretreatments can be mechanical, chemical, or biochemical processes or combinations thereof. The pretreatment can comprise one or more techniques including autohydrolysis, steam explosion, grinding, chopping, ball milling, compression mulling, radiation, flow-through liquid hot water treatment, dilute acid treatment, concentrated acid treatment, peracetic acid treatment, supercritical carbon dioxide treatment, alkali treatment, organic solvent treatment, and treatment with a microorganism, such as, for example a fungus or a bacterium. The alkali treatment can include sodium hydroxide treatment, lime treatment, wet oxidation, ammonia treatment, and oxidative alkali treatment. The pretreating can involve removing or altering lignin, removing hemicellulose, decrystallizing cellulose, removing acetyl groups from hemicellulose, reducing the degree of polymerization of cellulose, increasing the pore volume of lignocellulose biomass, increasing the surface area of lignocellulose, or any combination thereof.

### III. Enzymes

Cocktails of any combination of enzymes selected from enzymes including, but not limited to, the six major enzyme classifications of hydrolase, oxidoreductase, transferase, lyase, isomerase or ligase can be made (Nomenclature Committee of the International Union of Biochemistry and Molecular Biology (NC-IUBMB), Enzyme Nomenclature, Academic Press, San Diego, Calif., 1992). Examples of suitable enzymes include a cellulase, hemicellulase, xylanase, amylase, glucoamylase, protease, cutinase, phytase, laccase, lipase, isomerase, glucose isomerase, esterase, phospholipase, pectinase, keratinase, reductase, oxidase, peroxidase, phenol oxidase, lipoxygenase, ligninase, pullulanase, tannase, pentosanase, maltase, mannanase, glucuronidase, galactanase, β-glucanase, arabinosidase, hyaluronidase, lactase, polygalacturonase, β-galactosidase, and chondroitinase, or any enzyme for which closely related and less stable homologs exist.

The enzymes can be from any origin, e.g., bacteria or fungi. The enzymes can be a hybrid enzyme, i.e., a fusion protein which is a functional enzyme, wherein at least one part or portion is from a first species and another part or portion is from a second species. The enzymes can be a mutant, truncated or hybrid form of endogenous enzymes. The enzymes suitable for the present methods can be a secreted, cytoplasmic, nuclear, or membrane protein. Extracellular enzymes, e.g., a cellulase, hemicellulase, protease, or starch degrading enzyme such as amylase, usually have a signal sequence linked to the N-terminal portion of their coding sequence to facilitate secretion.

Examples of enzyme substrates include lignocellulosic materials, cellulose, hemicellulose, starch, or a combination thereof. An exemplary group of enzymes for catalyzing lignocellulosic materials conversion includes endoglucanases, exoglucanases, or cellobiohydrolases and β-glucosidases. An exemplary group of enzymes for catalyzing hemicellulose conversion includes at least xylanase, mannanase, xylosidase, mannosidase, glucosidase, arabinosidase, glucuronidase, and galactosidase. An exemplary group of enzymes for catalyzing starch hydrolysis include at least α-amylase, saccharifying α-amylase, β-amylase, glucoamylase, isoamylase, and pullulanase. Depending on the raw materials and pre-treatment methods, additional enzymes, e.g., proteases and phytases, can be selected.

Cellulases are enzymes that hydrolyze the β-D-glucosidic linkages in celluloses. Cellulolytic enzymes have been traditionally divided into three major classes: endoglucanases, exoglucanases or cellobiohydrolases and β-glucosidases (Knowles, J. et al., TIBTECH 5:255-261 (1987)). Cellulase enzymes also include accessory enzymes, including GH61 members, swollenin, expansin, and CIP1. Numerous cellulases have been described in the scientific literature, examples of which include: from Trichoderma reesei: Shoemaker, S. et al., Bio/Technology, 1:691-696, 1983, which discloses CBHI; Teeri, T. et al., Gene, 51:43-52, 1987, which discloses CBHII; Penttila, M. et al., Gene, 45:253-263, 1986, which discloses EGI; Saloheimo, M. et al., Gene, 63:11-22, 1988, which discloses EGII; Okada, M. et al., Appl. Environ. Microbiol., 64:555-563, 1988, which discloses EGIII; Saloheimo, M. et al., Eur. J. Biochem., 249:584-591, 1997, which discloses EGIV; and Saloheimo, A. et al., Molecular Microbiology, 13:219-228, 1994, which discloses EGV. Exo-cellobiohydrolases and endoglucanases from species other than Trichoderma have also been described, e.g., Ooi et al., 1990, which discloses the cDNA sequence coding for endoglucanase F1-CMC produced by Aspergillus aculeatus; Kawaguchi T. et al., 1996, which discloses the cloning and sequencing of the cDNA encoding β-glucosidase 1 from Aspergillus aculeatus; Sakamoto et al., 1995, which discloses the cDNA sequence encoding the endoglucanase CMCase-1 from Aspergillus kawachii IFO 4308; and Saarilahti et al., 1990, which discloses an endoglucanase from Erwinia carotovara.

Hemicellulases are enzymes that catalyze the degradation and/or modification of hemicelluloses, including xylanase, mannanase, xylosidase, mannosidase, glucosidase, arabinosidase, glucuronidase, and galactosidase. For example, the hemicellulase can be a xylanase, i.e., any xylan degrading enzyme which is either naturally or recombinantly produced. Generally, xylan degrading enzymes are endo- and exo-xylanases hydrolyzing xylan in an endo- or an exo-fashion. Exemplary xylan degrading enzymes include endo-1,3-β-xylosidase, endo-β1,4-xylanases (1,4-β-xylan xylanohydrolase; EC 3.2.1.8), 1,3-β-D-xylan xylohydrolase and β-1-4xylosidases (1,4-β-xylan xylohydrolase; EC 3.2.1.37) (EC Nos. 3.2.1.32, 3.2.1.72, 3.2.1.8, 3.2.1.37). Preferred xylanases are those which are derived from a filamentous fungus (e.g., the fungi of the genera Aspergillus, Disportrichum, Penicillium, Humicola, Neurospora, Fusarium, Trichoderma, and Gliocladium) or a bacterial source (e.g., Bacillus, Thetmotoga, Streptomyces, Microtetraspora, Actinmadura, Thermomonospora, Actinomyctes, and Cepholosporum).

Amylases are starch-degrading enzymes, classified as hydrolases, which cleave α-D-(1→4) O-glycosidic linkages in starch. Generally, α-amylases (E.C. 3.2.1.1, α-D-(1→4)-glucan glucanohydrolase) are defined as endo-acting enzymes cleaving α-D-(1→4) O-glycosidic linkages within the starch molecule in a random fashion. The exo-acting amylolytic enzymes, such as β-amylases (E.C. 3.2.1.2, α-D-(1→4)-glucan maltohydrolase), and some product-specific amylases like maltogenic alpha-amylases (E.C. 3.2.1.133), cleave the starch molecule from the non-reducing end of the substrate. β-Amylases, α-glucosidases (E.C. 3.2.1.20, α-D-glucoside glucohydrolases), glucoamylases (E.C. 3.2.1.3, α-D-(1→4)-glucan glucohydrolase), and product-specific amylases can produce malto-oligosaccharides of a specific length from starch.

Preferably, α-amylases are those derived from Bacillus sp., particularly those from Bacillus licheniformis, Bacillus amyloliquefaciens or Bacillus stearothermophilus, as well as Geobacillus stearothermophilus, and fungal α-amylases such as those derived from Aspergillus (e.g., A. terreus, A. kawachi, A. clavatus, A. oryzae, and A. niger). Optionally, α-amylases can be derived from a precursor α-amylase. The precursor α-amylase is produced by any source capable of producing α-amylase. Suitable sources of α-amylases are prokaryotic or eukaryotic organisms, including fungi, bacteria, plants or animals. Preferably, the precursor α-amylase is produced by Geobacillus stearothermophilus or a Bacillus; more preferably, by Bacillus licheniformis, Bacillus amyloliquefaciens, or Bacillus stearothermophilus; most preferably, the precursor α-amylase is derived from Bacillus licheniformis. α-Amylases can also be from Bacillus subtilis.

Glucoamylases are enzymes of amyloglucosidase class (E.C. 3.2.1.3, glucoamylase, 1,4-alpha-D-glucan glucohydrolase). These enzymes release glucosyl residues from the non-reducing ends of amylose and amylopectin molecules.

Pullulanases are starch debranching enzymes. Pullulanases are enzymes classified in EC 3.2.1.41 and such enzymes are characterized by their ability to hydrolyze the α-1,6-glycosidic bonds in, for example, amylopectin and pullulan.

Other enzymes include proteases, such as a serine, metallo, thiol or acid protease. Serine proteases (e.g., subtilisin) are described, for example, by Nedkov et al., Honne-Seylers Z. Physiol. Chem. 364:1537-1540, 1983; Drenth, J. et al. Eur. J. Biochem. 26:177-181, 1972; U.S. Pat. Nos. 4,760,025 (RE 34,606), 5,182,204, and 6,312,936; and EP 0 323,299. Proteolytic activity can be measured as disclosed in Kalisz, “Microbial Proteinases” Advances in Biochemical Engineering and Biotechnology, A. Fiecht, ed., 1988.

Phytases are enzymes that catalyze the hydrolysis of phytate to (1) myo-inositol and/or (2) mono-, di-, tri-, tetra- and/or penta-phosphates thereof and (3) inorganic phosphate. For example, phytases include enzymes defined by EC number 3.1.3.8, or EC number 3.1.3.26.

### IV. Cell Lines

Having selected a conversion process and identified from published literature and/or by experimentation one or more combinations of enzymes expected to enhance the conversion process, cell lines are identified or constructed to express different sets of the enzymes. The enzymes endogenously expressed by some cell lines are well known. For example, T. reesei is a source of several cellulose processing enzymes, Aspergillus is source of amylases, and Bacillus is a source of a number of amylases. Such cell lines are sometimes used without modification. Often, however, one or more enzymes desired to enhance the enzymatic conversion process are not endogenously expressed at sufficient levels by a known existing cell line. In this case, an existing cell line can be genetically engineered to express an enzyme exogenously. If several enzymes desired to enhance the conversion process are not expressed at sufficient levels by a known existing cell line, existing cell line(s) can be genetically engineered to express each of the enzymes exogenously. For maximum modularity, each such enzyme can be exogenously expressed in its own cell line. Preferably, the cell lines into which different enzymes are genetically engineered represent modifications of the same base cell line.

As a result of endogenous expression, exogenous expression, or both, cell lines to be co-cultured can express different sets or panels of enzymes, all of which contribute to the enhancement of enzymatic conversion. For a cell line that does not express any endogenous enzyme enhancing the conversion process, and which has been genetically engineered to express one or more exogenous enzymes, the set or panel of enzymes produced by the cell line are said to include exogenous enzyme(s). In a cell line that endogenously expresses enzyme(s) enhancing the conversion process, which has been genetically engineered to express one or more exogenous enzymes, the set or panel of enzymes produced by the cell line are said to include endogenous enzymes and exogenous enzymes. In a cell line that has not been genetically engineered to express an exogenous enzyme, the set or panel of enzymes produced by the cell line are said to include only endogenous enzymes. Although exogenous enzymes of a set are readily known and recognized, this is not necessarily the case for endogenous enzymes expressed at trace levels. For this reason, the set or panel of enzymes is defined as including only enzymes expressed at detectable levels as determinable by HPLC according to the conditions and/or protocols used in the examples. Preferably each enzyme in a set is expressed at a level of at least 1/100 or 1/10 the level of the most highly expressed enzyme in the set. For example, when the expressed enzyme is secreted, the level of the expression can be determined relative to the amount of enzyme that is secreted. It is not necessary for practice of the present methods to know the identity of all enzymes falling within a set. Rather, it is sufficient to know the identity of at least one enzyme within a set produced by a given cell line.

The set of enzymes encoded by one cell line can contain no, partial, or complete overlap with the set of enzymes encoded by a second cell line. Enzymes present in the first set of the first cell line and enzymes present in the second set of the second cell line may be expressed at different levels. If the identities of the enzymes in the sets completely overlap, then at least one enzyme is expressed at a different level (i.e., the standard errors of means (SEMs) do not overlap) between the sets. Preferably, each set of enzymes includes at least one enzyme not expressed or expressed at a lower level in other set(s) of enzymes from other cell line(s) included in the co-culture. Preferably at least one enzyme in one set of enzymes (e.g., a first set) catalyzing a conversion process is exogenous to that cell line expressing the same set of enzymes (e.g., a first cell line). Preferably a co-cultured cell line expresses an endogenous enzyme, which is otherwise not expressed or expressed at significantly lower levels by each other cell line included in the co-culture. When one set of enzymes includes an exogenous enzyme and all enzymes in other sets of enzymes are endogenous, the cell lines expressing the other sets can be a strain, a species, or a genus different than that of the first cell line. Alternatively, one cell line can be a base strain or cell line modified to express an exogenous enzyme, and another cell line can be the base cell line or strain without the modification. Although it might be thought that co-expression of the modified cell line with the base cell line would undesirably dilute the relative concentration of exogenous enzyme relative to endogenous enzymes produced by the base cell line, in fact, the modification may substantially suppress expression of an endogenous enzyme that would otherwise enhance the conversion process. In this situation, co-cultivation of the modified cell line with the base strain or cell line can provide a blend of the exogenous and endogenous enzymes in more effective proportions than culture of either cell line alone.

By co-culturing two or more cell lines, different sets of enzymes can be expressed together, achieving ratios of enzymes or enzymatic activities different than those of each cell line alone. The ratios are preferably by moles, but activity units, mass, or other units also can be used.

The ratio of any enzymes can be compared by assessing the difference between (1) a first set of enzymes and a second set of enzymes in a mixture of enzymes resulting from co-culture and (2) one or both individual cell lines. Such a comparison is most readily illustrated on a pair-wise basis between the most highly expressed enzyme in the first set and the most highly expressed enzyme in the second set (expression being measured at the protein level, preferably of a secreted protein). The ratio of such enzymes in either individual cell line is preferably at least 2, 3, 4, 5, 10, 15, 20, 25, 30, 35, 40, 45, or 50-fold different than in the mixture of enzymes. For example, if the highest expressed enzyme in a first set and the highest expressed enzyme in a second set are expressed at a 1:1 molar ratio in a mixture resulting from co-culture and a 10:1 ratio in a first cell line and a 1:10 ratio in a second cell line, then the molar ratio is 10-fold different in the mixture than either cell line. Pair-wise or group comparisons can be made between any other enzymes in the first or second set. A group used for a comparison can be defined as, e.g., secreted enzymes in each set, intracellular enzymes in each set, exogenous enzymes in each set, or enzymes having a recombinant tag in each set.

Cell lines are engineered to express one or more exogenous enzymes by conventional methods. Representative engineered host cells (e.g., A. niger), expression vectors, promoters, and recombinant engineering procedures for expressing exogenous enzymes (e.g., glucoamylase or variants thereof) are disclosed in U.S. Pat. No. 8,426,183, for example. In some such methods, a nucleic acid encoding an enzyme in operable linkage to regulatory sequences to ensure its expression is transformed into the cell line. Optionally the enzyme can be fused to a recombinant tag (e.g., His-tag, FLAG-tag, GST, HA-tag, MBP, Myc-tag) to facilitate detection or quantification in co-culture or in a mixture of enzymes resulting from co-culture. The nucleic acid encoding the enzyme is preferably also fused to a signal peptide to allow secretion. Any suitable signal peptide can be used depending on the enzyme to be expressed and secreted in a host organism. Examples of signal sequences include a signal sequence from a Streptomyces cellulase gene. A preferred signal sequence is a S. lividans cellulase, celA (Bently et al., Nature 417:141-147, 2002). The nucleic acid is then preferably stably maintained either as a result of transformation on an episome or through integration into the chromosome. Alternatively, expression of an enzyme can be induced by activating in cis or in trans DNA encoding the enzyme in the chromosome.

As well as engineering cell lines to express an exogenous gene, it is sometimes desirable to engineer cell lines to inhibit or knockout expression of an endogenous gene encoding a product that is an inhibitor to the conversion process. The inhibition or knockout strategy can also be used to remove unnecessary genes or replacing an endogenous gene and replacing it with an improved version, a variant of, and/or a heterologous version of that gene. Such inhibition or knockout can be performed by siRNA, zinc finger proteins, other known molecular biology techniques used to knockout or reduce expression of particular endogenous genes, or the like.

The cell lines combined for co-culture can be from different, or same, domains, kingdoms, phylums, classes, subclasses, orders, families, genera, or species. They can also be from different strains of different species, different strains of the same species, or from the same strain.

Exemplary combinations include cell lines from different strains of the same species (e.g., T. reesei RL-P37 (Sheir-Neiss et al., Appl. Microbiol. Biotechnol. 20:46-53, 1984) and T. reesei QM-9414 (ATCC No. 26921), isolated by the U.S. Army Natick Laboratory). Cell lines from different strains of different species in the same kingdom (e.g., fungus) can be used (e.g., T. reesei RL-P37 and Aspergillus niger). Cell lines from different strains of different species in different kingdoms/domains can also be used (e.g., bacteria, yeast, fungi, algae, and higher eukaryotic cells (plant or animal cells)). Exemplary combinations further include a bacterium (e.g., B. subtilis or E. coli) and a fungus (e.g., T. reesei or Aspergillus niger); a bacterium and a yeast (e.g., Saccharomyces or Pichia); a yeast and a fungus; a bacterium and an algae, a yeast and an algae, a fungus and an algae, and so forth.

When two or more cell lines are engineered from a same base strain (e.g., T. reesei, RL-P37, or B. subtilis), each cell line can encode one or more different exogenous enzymes. Optionally, some cell lines can also be engineered so that a gene in the base strain is suppressed or inhibited, e.g., by at least 50%, 75%, or 90%, of the normal expression level.

The cell lines suitable for the present methods include bacteria, yeast, fungi and higher eukaryotic cell lines such as plant or animal cell lines. Microbial cell lines are preferred.

The cell lines can be yeast cell lines. Examples of yeast cells include Saccharomyces sp., Schizosaccharomyces sp., Pichia sp., Hansenula sp., Kluyveromyces sp., Prtaffia sp., or Candida sp., such as Saccharomyces cerevisiae, Schizosaccharomyces pombe, Candida albicans, Hansenula polymorpha, Pichia pastoris, P. canadensis, Kluyveromyces marxianus, and Phaffia rhodozyma.

The cell lines can be fungal cell lines. Examples of fungi include species of Aspergillus such as A. oryzae and A. niger, species of Saccharomyces, such as S. cerevisiae, species of Schizosaccharomyces, such as S. pombe, and species of Trichoderma, such as T. reesei.

Preferred examples of fungi include filamentous fungal cells. The filamentous fungal parent cell may be a cell of a species of, but not limited to, Trichoderma (e.g., Trichoderma reesei, the asexual morph of Hypocrea jecorina, previously classified as T. longibrachiatum, Trichoderma viride, Trichoderma koningii, Trichoderma harzianum) (Sheir-Neiss et al, Appl. Microbiol. Biotechnol. 20: 46-53, 1984; ATCC No. 56765 and ATCC No. 26921); Penicillium sp.; Humicola sp. (e.g., H. insolens, H. lanuginose, or H. grisea); Chrysosporium sp. (e.g., C. lucknowense); Gliocladium sp.; Aspergillus sp. (e.g., A. oryzae, A. niger, A sojae, A. japonicus, A. nidulans, or A. awamori) (Ward et al., Appl. Microbiol. Biotechnol. 39: 7380743, 1993 and Goedegebuur et al., Genet. 41: 89-98, 2002); Fusarium sp. (e.g., F. roseum, F. graminum, F. cerealis, F. oxysporuim, or F. venenatum); Neurospora sp. (e.g., N. crassa); Hypocrea sp.; Mucor sp. (e.g., M. miehei); Rhizopus sp.; and Emericella sp. (see Innis et al, Science 228: 21-26, 1985). The terms “Trichoderma,” “Trichoderma sp.,” or “Trichoderma spp.” refer to any fungal genus previously or currently classified as Trichoderma. The fungus can be A. nidulans, A. awamori, A. oryzae, A. aculeatus, A. niger, A. japonicus, T. reesei, T. viride, F. oxysporum, or F. solani. Aspergillus strains are disclosed in Ward et al., Appl. Microbiol. Biotechnol. 39:738-743, 1993 and Goedegebuur et al., Curr. Gene 41:89-98, 2002, which are each hereby incorporated by reference in their entirety, particularly with respect to fungi. Preferably, the fungus is a strain of Trichoderma, such as a strain of T. reesei. Strains of T. reesei are known and non-limiting examples include ATCC No. 13631, ATCC No. 26921, ATCC No. 56764, ATCC No. 56765, ATCC No. 56767, and NRRL 15709, which are each hereby incorporated by reference in their entirety, particularly with respect to strains of T. reesei. The host strain can be a derivative of RL-P37 (Sheir-Neiss et al., Appl. Microbiol. Biotechnol. 20:46-53, 1984).

The cell lines can be bacterial cell lines. Examples of bacterial cells suitable for the present methods include a gram-positive bacterium (e.g., Streptomyces and Bacillus) and a gram-negative bacterium (e.g., Escherichia coli and Pseudomonas sp.). Preferred examples include strains of Bacillus, such as B. licheniformis or B. subtilis, strains of Lactobacillus, strains of Streptococcus, strains of Pantoea, such as P. citrea, strains of Pseudomonas, such as P. alcaligenes, strains of Streptomyces, such as S. albus, S. lividans, S. murinus, S. rubiginosus, S. coelicolor, or S. griseus, or strains of Escherichia, such as E. coli. The genus “Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

The cell lines can be plant cell lines. Examples of plant cells include a plant cell from the family Fabaceae, such as the Faboideae subfamily. Examples of plant cells suitable for the present methods include a plant cell from kudzu, poplar (such as Populus alba×tremula CAC35696 or Populus alba) (Sasaki et al., FEBS Letters 579(11): 2514-2518, 2005), aspen (such as Populus tremuloides), or Quercus robur.

The cell lines can be an algae cell, such as a green algae, red algae, glaucophytes, chlorarachniophytes, euglenids, chromista, or dinoflagellates.

The cell lines can be a cyanobacteria cell, such as cyanobacteria classified into any of the following groups based on morphology: Chroococcales, Pleurocapsales, Oscillatoriales, Nostocales, or Stigonematales.

The cell lines can be a mammalian cell such as Chinese hamster ovary (CHO) cells, HeLa cells, baby hamster kidney (BHK) cells, COS cells, or any number of other immortalized cell lines available from the American Type Culture Collection, for example.

In some methods, the first cell line is a T. reesei strain encoding an exogenous β-xylosidase, and the second cell line is a T. reesei strain encoding an exogenous β-glucosidase.

In some methods, the first cell line is a B. licheniformis strain encoding Bacilllus licheniformis amylase, and the second cell line is a B. licheniformis strain encoding Geobacillus stearothermophilus amylase.

In some methods, for example, the first cell line is a T. reesei strain encoding an exogenous GH61 enzyme, and the second cell line is a T. reesei strain encoding exogenous or endogenous cellulases.

### V. Co-Culturing Methods

The cell lines to be co-cultured can in some embodiments be separately cultured initially to form starter cultures, which preferably have an optical density of at least about 0.1, 0.2, 0.4, 0.8, 1.0, or 1.5 at a wavelength of 600 nm and a path length of 1 cm. The starter cultures are then mixed in equal volumes or other desired ratio (as discussed further below) in fresh culture media to form a starting co-culture. Optionally, isolates can be directly inoculated in culture media for protein production (e.g., without the use of starter cultures).

Potential issues of one cell line outgrowing another can be reduced by selecting cell lines, e.g., closely related cell lines, with inherently similar growth characteristics, selecting a culture media that is not optimal for at least one of the cell lines but reduces differences in growth when each cell line is grown on separate culture media, and/or adjusting the ratio by volume, OD, or cell count, with which cultures are combined to compensate for different growth characteristics.

Closely related cell lines may be obtained from the same species (e.g., T. reesei) or the same strain, or more preferably the same base strain or cell line modified in different ways to express different exogenous enzymes. For example, a first cell line may be a base cell line genetically engineered to express enzyme A, and a second cell line may be the base cell line genetically engineered to express enzyme B.

Before combining the cell lines for co-culturing, the growth profile of each cell line can be determined. Based on the determined growth profiles, a ratio or a range of ratios, with which to mix the cell lines for optimal co-expression of the first set of enzymes and the second set of enzymes (or more sets of enzymes), can then be determined to compensate at least in part for differences in growth profiles.

In any cell culture system, there is a characteristic growth pattern following inoculation that includes a lag phase, an accelerated growth phase, an exponential or “log” phase, a negative growth acceleration phase, and a plateau or stationary phase. The log and plateau phases give information about the cell line, the population doubling time during log growth, the growth rate, and the maximum cell density achieved in plateau. For example, in the log phase, as growth continues, the cells reach their maximum rate of cell division, and numbers of cells increase in log relationship to time. By making a first count at a specified time and a second count after an interval during the log phase and knowing the number of elapsed time units, one can calculate the total number of cell divisions or doublings, the growth rate and generation time.

Measurement of the population doubling time can be used to monitor the culture during serial passage and calculate cell yields and the dilution factor required at subculture. The population doubling time is an average figure and describes the net result of a wide range of cell division rates within the culture. The doubling time differs with varying cell types, culture vessels, and conditions. Pre-determined growth profiles can be used to determine the population doubling time for each cell line used in the co-culture. Preferably, the population doubling times in exponential growth of cell lines to be co-cultured are within a factor of 2 or 5 of each other. For example, the population doubling time in exponential growth of cell lines selected to be co-cultured are within a factor of 2, 3, 4, or 5 of each other. If the growth rates differ more broadly, then the culture media is preferably varied to identify a culture media on which the population doubling times are more similar, preferably within a factor of 2 or 5 of each other. For example, the components and conditions provided by the culture media can be adjusted and used to reduce the differences in population doubling time in exponential growth of cell lines such that the population doubling times for each cell lines become within a factor of 2 or 5 of each other. Additionally, cell lines can first be selected based on their small differences in growth profiles using conventional culture media, followed by adjustment of culture media/conditions, such that the growth profile differences become even smaller.

The optimal ratio of sets of enzymes encoded by a first cell line to a second cell line is not necessarily known a priori. Combination of the cell lines in different ratios by volume, OD, or number of cells allows different ratios to be compared empirically on a small scale, with an optimal ratio identified by such analysis being used for subsequent larger scale culture.

To ensure no single cell line unacceptably outcompetes one or more other cell lines, e.g., by growing more rapidly and suppressing the growth of other cell lines, the ratio of cell lines can be adjusted so that each cell line reaches a defined point in the growth curve at about the same time. For example, the ratio can be adjusted so each cell line reaches mid-log phase at about the same time. Alternatively, each cell line can reach plateau phase (mid-plateau phase) at about the same time. Preferably, each cell line can reach both the mid-log phase and the plateau phase at about the same time. Optionally, each cell line can reach stationary phase at about the same time.

The growth profiles can also be used to determine the harvest time and/or seeding densities required for achieving certain ratios of harvesting cell densities between/among the cell lines. For example, an equal molar ratio of different sets of enzymes may be desired for one type of substrates/pretreatment methods. Different ratios of enzymes desired for other types of substrates/pretreatment methods can be achieved by varying the seeding densities of one or more cell lines as well as the harvest time.

Each cell line can have different requirements for optimal growth in culture media, particularly for cell lines from different organisms (e.g., different domains, kingdoms, genera, or species), or different strains. However, a culture media, although not optimal for any single cell line, can be optimal for co-fermentation of all cell lines if all cell lines have similar growth profiles in such a media. Accordingly, the growth profile of each cell line in multiple culture media can be determined. These growth profiles are then compared to identify a culture media in which the growth profiles of the cell lines are the most similar. For example, in such a media each cell line reaches plateau phase (mid-plateau phase), mid-log phase, and/or stationary phase at about the same time. The chosen culture media is then used for co-culture.

As an alternative to, or in combination with, the cell density-based growth profiling, the amounts of the enzymes and/or the activities of the expressed enzymes can be measured along the growth curve. These variations along the growth curve provide guidance for determining the ratio with which to mix the cell lines for optimal co-expression of the enzymes. For example, the expression levels of some enzymes may be lower than other enzymes. For these enzymes, a higher seeding density of the cell lines expressing the enzymes is preferred to achieve a desired amount of these low expressed enzymes.

Cell lines from the same strain usually have similar growth profiles and require similar culture media. On the other hand, cell lines from different strains or different organisms often have different growth profiles and require different culture media. As discussed above, growth profiles of different cell lines can be measured to determine the seeding density for each cell line. Optionally, growth profiles in various culture media for each cell line are measured to determine a media suitable for co-culture.

The enzymes can be released directly to the culture media. Alternatively cells can be lysed releasing intracellular enzymes. Furthermore, some enzymes expressed by a given cell line can be released directly whereas other enzymes may be released by cell lysis. The released enzymes, whether as a result of secretion or lysis, can be harvested from the culture media, or the culture medium can be used as is with minimal if any further processing as a whole broth. Cell debris (e.g., host cells, lysed fragments), can optionally be removed by, e.g., centrifugation or ultrafiltration if desired. Optionally, the enzyme mixture can be concentrated, e.g., with a commercially available protein concentration filter. The enzyme mixture can be separated further from other impurities by one or more purification steps, e.g., immunoaffinity chromatography, ion-exchange column fractionation (e.g., on diethylaminoethyl (DEAE) or matrices containing carboxymethyl or sulfopropyl groups), chromatography on Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, or protein A Sepharose, SDS-PAGE chromatography, silica chromatography, chromatofocusing, reverse phase HPLC (RP-HPLC), gel filtration using, e.g., Sephadex molecular sieve or size-exclusion chromatography, chromatography on columns that selectively bind the peptide, and ethanol, pH or ammonium sulfate precipitation, membrane filtration and various techniques. In some methods, the enzyme mixture is used in downstream application with minimal, if any, further processing.

The amounts of the enzymes secreted or lysed from cells or in finished product can be measured using conventional techniques, e.g., by reverse phase high performance liquid chromatography (RP-HPLC), or sodium dodecyl sulfate-polyacrylamide gel electrophoresis (SDS-PAGE). The activities of the enzymes can also be measured using methods well-known in the art.

### VI. Cell Banking

Cell lines expressing different sets of enzymes can be stored in a cell bank and co-cultured in different combinations. A cell bank can be constructed with a particular conversion process or a particular set of conversions in mind. Enzymes enhancing the conversion process are identified either from known or published sources, from experiments, or from both. Cell lines are then identified or constructed encoding and disposed to express different sets of the enzymes. Cell lines expressing one or more of the enzymes endogenously or exogenously may already be known. Cell lines expressing one or more of the enzymes exogenously can also be constructed particularly if no cell line expressing a particular enzyme or a particular combination or panel of enzymes at sufficient levels is already available.

The cell lines in a bank can be stored on solid or liquid media in the cold or frozen. Before use, a vial of cells is typically separately propagated to form a starter culture, which can also take place in a liquid or on solid medium. The cell lines can be propagated and stored under different selective pressures to retain expression of the respective sets of enzyme and avoid the possibility of cross contamination. Alternatively, the cell lines can be propagated and stored under conditions that allow growth of the auxotrophs, thereby maintaining the genotypes.

The cell lines can be co-cultured and used to prepare a mixture of enzymes using the methods described above. After combination, the cell lines are propagated on media in which the combined cell lines are used, so selective conditions or conditions that allow auxotroph growth that may have been used for separate propagation and storage of the cells lines are not necessarily used in the co-culture step.

The cell bank may allow selection of different permutations of cell lines that provide enzymes enhancing the conversion process in different combinations or relative expression levels. The different combinations can be compared to determine which given enzyme mixture has the best activity for enhancing the conversion process. Such a comparison can indicate the best combination of cell lines within a bank without necessarily knowing a priori exactly which enzymes or what ratio of enzymes is optimal. In that sense, this allows the tailoring of the panel of expressed enzymes from a co-culture to the particular requirements of a particular conversion process.

Variations in the substrates or pretreatment of substrates for a different process can be accommodated by varying the ratio in which starter cultures of cell lines from the cell bank are combined. For example, the amount of hemicellulose may vary in a cellulose preparation. Enzyme cocktails for treating high amounts of hemicellulose can contain a higher level of xylanase activity. Some starch preparations may contain a substance (e.g., raw material or metabolite) known to be inhibitory of amylase activity, in which case a higher amylase amount is desirable. Depending on the compositions (e.g., different glucan/xylan profile) in the pre-treated substrates, different enzyme cocktails can be prepared by mixing starter cultures of enzyme production strains in different ratios, thereby producing enzyme cocktails having different relative amounts of the enzymes.

Cell banking can also be useful irrespective of the conversion process. By banking different cell lines encoding a variety of commonly used industrial enzymes, the cell lines can be combined in different combinations from the bank for co-culture depending on the conversion process at hand. The co-fermentation method provided herein therefore not only provides flexibility of a resulting composition, but also affords various other advantages such as, for example, reduced costs as compared to conducting fermentation of each desired enzyme component separately followed by blending; reduced cost for storage of enzymes because co-fermentation results in a composition with desired ratios of enzymes, whereas the blending strategy will require storage for each individual enzyme separately fermented or prepared.

### VII. Applications

The enzyme mixtures produced by the present methods have various agricultural, industrial, medical and nutritional applications where such a conversion process is utilized. The substrates of such a conversion process may comprise lignocellulosic materials, cellulose, hemicellulose, and starch.

For example, a mixture of cellulase enzymes and/or cellulase accessory enzymes can be used for hydrolysis of cellulolytic materials, e.g., in the fermentation of biomass into biofuels. The mixture is also useful for generating glucose from grain, or as a supplement in animal feed to decrease the production of fecal waste by increasing the digestibility of the feed. Cellulase enzymes can also be used to increase the efficiency of alcoholic fermentations (e.g., in beer brewing) by converting lignocellulosic biomass into fermentable sugars. The cellulase mixture can be used for commercial food processing in coffee, i.e., hydrolysis of cellulose during drying of beans. They have also been used in the pulp and paper industry for various purposes. In pharmaceutical applications, cellulases are useful as a treatment for phytobezoars, a form of cellulose bezoar found in the human stomach.

A mixture of cellulase enzymes, cellulase accessory enzymes, and/or hemicellulase enzymes are widely used in textile industry and in laundry detergents. Cellulases can also be used in hydrolyzing cellulosic or lignocellulosic materials into fermentable sugars.

A mixture of amylases or a mixture of α-amylase, β-amylase, glucoamylase, and/or pullulanases has various applications in the food industry. For example, a mixture of amylase enzymes is useful in syrup manufacture, dextrose manufacture, baking, saccharification of fermented mashes, food dextrin and sugar product manufacture, dry breakfast food manufacture, chocolate syrups manufacture, and starch removal from fruit juices. Amylases can also be used in producing glucose from grain products for ethanol production.

A mixture of enzymes containing phytases can be used in grain wet milling and cleaning products. They also find many other uses in personal care products, medical products and food and nutritional products, as well as in various industrial applications, particularly in the cleaning, textile, lithographic and chemical arts.

### Examples

The following example describes representative Simultaneous Saccharification and Co-Fermentation (SSCF) reactions conducted under various conditions. Whole ground corn liquefact and corn flour are used as exemplary substrates.

### (A) Materials:

The following enzymes were obtained:
Purified Trichoderma reesei glucoamylase (TrGA) (DuPont Industrial Biosciences, Palo Alto, Calif.) at 0.054% w/w;
GC626 (a starch hydrolyzing alpha-amylase derived from Aspergillus kawachii and expressed in Trichoderma reesei) (DuPont Industrial Biosciences, Palo Alto, Calif.) at 0.0053% w/w; and
FERMGEN™ 2.5× (a fungal protease) (DuPont Industrial Biosciences, Palo Alto, Calif.) at 0.00029% w/w.

Whole ground corn liquefact at 32.8% dry solids (ds) for conventional simultaneous saccharification and fermentation (SSF) was obtained from a typical dry grind ethanol facility. The yeast strain used was Ethanol Red® Saccharomyces cerevisiae yeast (Fermentis, France).

The following glucoamylase-secreting fungal strains were used:
Trichoderma reesei fungal strain; and
Aspergillus niger fungal strain.

### (B) Conventional Fermentation Method:

A 100 gram conventional fermentation was conducted as follows. Frozen liquefact was incubated at 4° C. overnight. Following 4° C. incubation, the liquefact was incubated at 60° C. for 2 hours, followed by incubation at 32° C. for 30 minutes. Corn liquefact was weighed, and urea was added to a final concentration of 600 parts per million (ppm). The liquefact pH was adjusted to 4.8, using 6N sulfuric acid and/or 28% ammonium hydroxide. The solution was mixed well with an overhead stirrer for 30 minutes at room temperature. 100 g+/−0.2 g of liquefact was weighed out into individually labeled 125 mL Erlenmeyer flasks in replicates of three.

In the appropriate flasks, 100 g of the above liquefact were seeded with the appropriate fungal strain from A. niger or T. reesei and incubated at 32° C. for 9 hours with mixing at 200 rpm. Following seed incubation, at SSF time=0, a slurry of active dry yeast (ADY) in Milli-Q™ (Millipore Corp., Billerica, Mass.) water was prepared at a 20% yeast dose. Flasks were pitched, i.e., inoculated, with 0.5 mL hydrated yeast slurry. A second fungal inoculation procedure was performed by eliminating the seed period and pitching the A. niger or T. reesei fungal strain directly with the yeast at SSF time=0.

At SSF time=0, an appropriate volume of glucoamylase, GC626, and FERMGEN™ 2.5× were added to the appropriate flasks. Purified glucoamylase was dosed at 0.054% w/w. GC626 was dosed 0.0053% w/w. FERMGEN™ 2.5× was dosed at 0.00029% w/w. Each flask was mixed and stoppered with a foam stopper. The flasks were incubated in a forced-air incubator with mixing at 200 rpm for 55 hours at three separate temperature profiles. Approximately 1 mL time point samples were collected before and during SSF. Samples were stored frozen.

### (C) Sample Preparation Method:

Each time point sample was thawed at 4° C. and centrifuged at 15,000 rpm for 2 to 4 minutes. In individual wells of a 96-well deepwell microtiter plate, 100 μL sample supernatant were mixed with 10 μL 1.1 N sulfuric acid and incubated at 100° C. for 5 minutes. 1 mL of Milli-Q™ water was added to each well, and 200 μL of each sample was transferred into a 0.22 μm filter plate. Each sample was filtered into a separate 96 well microtiter plate. Each plate was sealed with an EZ-Pierce™ plate seal (Excel Scientific, Inc., Victorville, Calif.).

20 μL of each sample were loaded onto an Agilent 1200 series HPLC (Agilent Technologies, Inc., Santa Clara, Calif.) and analyzed using a Rezex™ RFQ-Fast Acid H+(8%) column (Phenomenex, Torrance, Calif.) at 85° C., 0.01 N sulfuric acid mobile phase at 1 mL/min, and a 9 minute elution time with a Rezex™ Organic Acid ROA guard column (Phenomenex, Torrance, Calif.) and a refractive index detector (RID) set at 55° C.

DP1, DP2, DP3, DP4+, glycerol, acetic acid, lactic acid, and ethanol concentrations (% w/v) were calculated using ChemStation (Agilent Technologies, Inc., Santa Clara Calif.) with appropriate calibration curves. Calibration curves for the above components were prepared at 1:1, 1:2, 1:5, 1:10, and 1:20 dilutions using a Supelco Fuel Ethanol Standard (Sigma Catalog #48468-U). 100 μL of each dilution was mixed with 10 μL 1.1 N sulfuric acid and 1 mL Milli-Q™ water and run as controls on the ChemStation system.

### Results

TABLE 1 describes the various blends and experimental conditions that were tested and described in more detail below:

TABLE 1

| | | | Exogenous | | | |
|---|---|---|---|---|---|---|
| | Exogenous GA | Exogenous | FERMGEN ™ | | | Seed |
| | Dose | GC626 Dose | 2.5x Dose | Fungal | Temperature | Time |
| Blend Name | (% w/w) | (% w/w) | (% w/w) | Strain | (° C.) | (hours) |
| Control 32° C. | 0.054 | 0.0053 | 0.00029 | N/A | 32 | 0 |
| Control 35° C. | 0.054 | 0.0053 | 0.00029 | N/A | 35 | 0 |
| Control 38° C. Ramp | 0.054 | 0.0053 | 0.00029 | N/A | 32-38-32 | 0 |
| Negative Control 32° C. | 0 | 0 | 0 | N/A | 32 | 0 |
| Negative Control 35° C. | 0 | 0 | 0 | N/A | 35 | 0 |
| Negative Control 38° C. Ramp | 0 | 0 | 0 | N/A | 32-38-32 | 0 |
| GC626 Negative Control | 0 | 0.0053 | 0.00029 | N/A | 32-38-32 | 0 |
| 1 | 0 | 0 | 0 | A. niger | 32 | 9 |
| 3 | 0 | 0.0053 | 0.00029 | T. reesei | 32 | 9 |
| 5 | 0 | 0 | 0 | A. niger | 35 | 9 |
| 7 | 0 | 0.0053 | 0.00029 | T. reesei | 35 | 9 |
| 10 | 0 | 0 | 0 | A. niger | 32-38-32 | 9 |
| 12 | 0 | 0.0053 | 0.00029 | T. reesei | 32-38-32 | 9 |
| 13 | 0 | 0 | 0 | A. niger | 32-38-32 | 0 |
| 14 | 0 | 0.0053 | 0.00029 | T. reesei | 32-38-33 | 0 |

TABLE 1 shows the blends using yeast without exogenous enzymes as the three negative controls. The addition of exogenous enzymes is required for efficient completion of an SSF run in the absence of added filamentous fungi expressing enzymes, e.g., A. niger or T. reesei. Fermentation to ethanol is very slow in the negative controls, producing an average of only 18% of the total ethanol yield produced from the positive control, as shown in TABLE 2. A fourth negative control blend (“GC626 negative control”) was run using only exogenous GC626 and FERMGEN™ 2.5× but no exogenous GA. Although the production of ethanol is higher in blends containing only GC626 and FERMGEN™ 2.5×, the final ethanol yield for the GC626 negative control is only 30% of the total yield under traditional SSF conditions, as shown in TABLE 2.

Without the addition of exogenous enzymes, a considerable amount of DP4+ is left after 55 hours regardless of the temperature used for the reaction. The amount of DP4+ left in the reaction under the various reaction conditions and the fold-increase in DP4+ relative to the control reactions are shown in TABLE 2.

TABLE 2

| | % Ethanol | DP4 + | % Total DP4 + |
|---|---|---|---|
| Blend | Yield | x-fold | Hydrolysis |
| Control 32° C. | 100% | 1 | 97% |
| Negative Control 32° C. | 19% | 35.5 | 2% |
| 1 | 86% | 2.19 | 94% |
| 3 | 53% | 9.67 | 73% |
| Control 35° C. | 100% | 1 | 97% |
| Negative Control 35° C. | 17% | 33.31 | 3% |
| 5 | 92% | 1.4 | 96% |
| 7 | 36% | 9.41 | 73% |
| Control 38° C. Ramp | 100% | 1 | 97% |
| Neg. Control 38° C. Ramp | 18% | 31.5 | 3% |
| GC626 Neg. Control | 30% | 10.7 | 63% |
| 10 | 99% | 1.28 | 96% |
| 12 | 28% | 9.73 | 67% |
| 13 | 98% | 1.09 | 96% |
| 14 | 30% | 14.5 | 50% |
| Control 32° C. | 100% | 1 | 97% |
| Control 35° C. | 96% | 1.05 | 97% |
| Control 38° C. Ramp | 90% | 1.11 | 97% |

To reduce or eliminate the need for the addition of exogenous enzymes, e.g., glucoamylase, co-fermentation of a fungal strain expressing GA and/or AA with yeast can provide the needed enzymes to catalyze the hydrolysis of starch to glucose. Two different fungal strains expressing GA and AA or GA were tested: Aspergillus niger and Trichoderma reesei. A. niger is capable of co-expressing both an endogenous GA and an acid stable alpha-amylase (AsAA). T. reesei expresses its endogenous GA without expressing significant levels of an alpha-amylase.

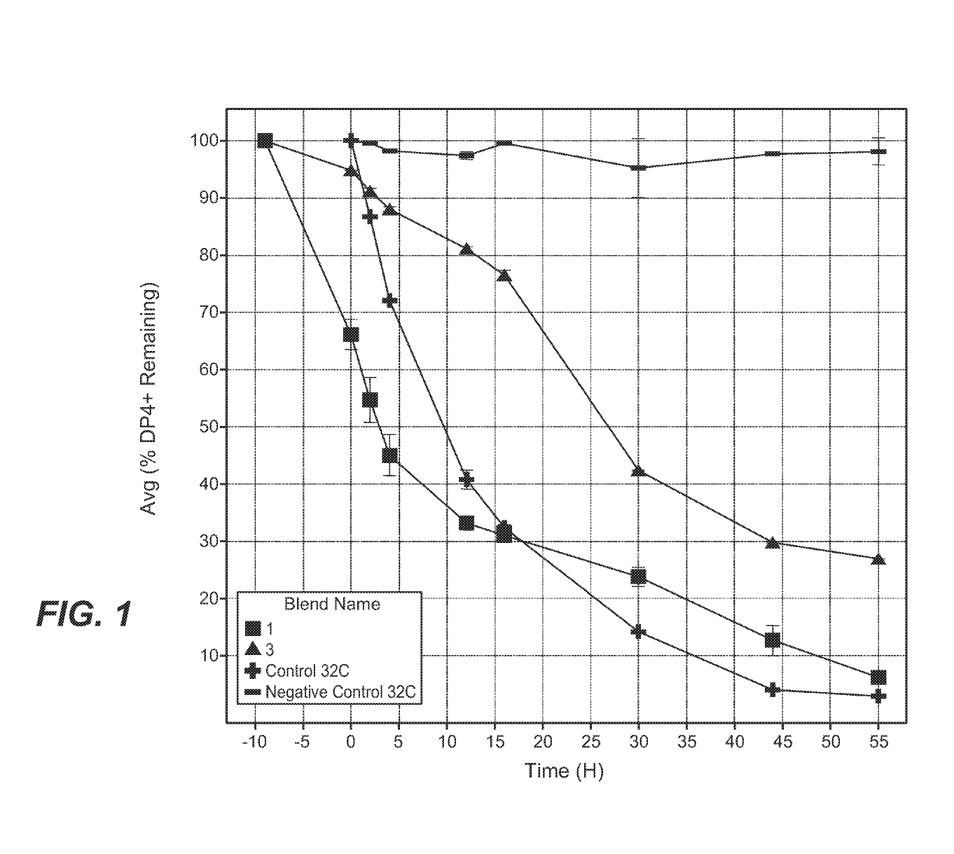

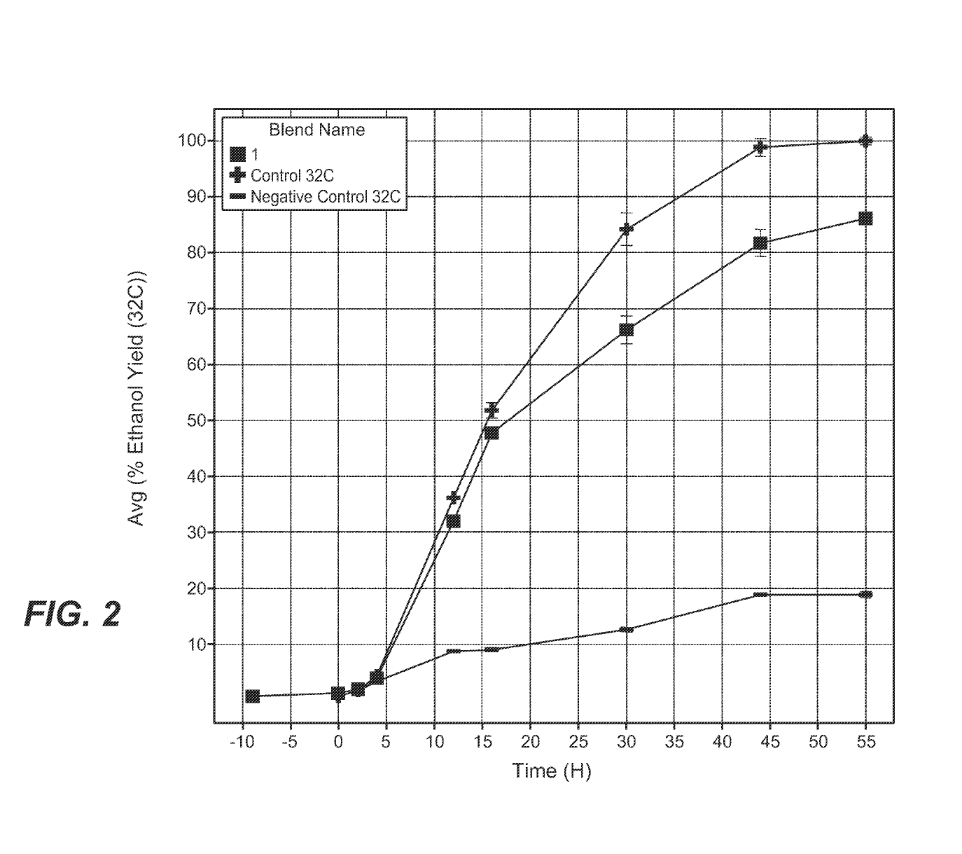

The addition of filamentous fungi, e.g., A. niger or T. reesei, to the SSCF conversion process decreases DP4+ levels, compared to the negative controls. A time course of DP4+ hydrolysis at 32° C. without exogenous GA (blends 1 and 3) and with exogenous AA (blend 3), for example, is depicted in FIG. 1. Similar time courses were seen under the other experimental conditions. The addition of filamentous fungi, specifically A. niger, to the SSCF conversion process increases the amount of ethanol production, even without the addition of exogenous enzymes. FIG. 2, for example, depicts total ethanol production after a 9 hour seed incubation and 55 hour SSCF of A. niger (blend 1) versus the controls under conventional fermentation conditions at 32° C.

Under typical industrial operating conditions, SSFs are run at 32° C. as an optimal temperature condition for yeast growth. During fermentation, however, temperatures can exceed 32° C. and reach as high as 38° C. To account for the variability in SSF temperature, the experiments were performed at three temperature profiles: 32° C., 35° C., and a ramped condition ranging from 32° C. to 38° C. with the maximum temperature peaking around 20 hours into fermentation and returning to 32° C. around 40 hours into fermentation. FIG. 3 shows representative temperature conditions used during fermentation.

Temperature significantly impacts the growth and viability of the yeast and thus impacts total ethanol produced throughout fermentation. As temperature increases, the yeast start to undergo stress and either die or suffer slower metabolism, leading to high levels of residual glucose (DP1) and lower ethanol yields. Ethanol yields at end of fermentation (EoF) are 10% lower under the highest stress 38° C. staged condition with exogenous TrGA and GC626 alpha-amylase, compared to the yields reached at 32° C., as shown in TABLE 2.

To increase enzyme expression and thereby promote the efficiency of SSF, the fungal strains may be pre-incubated in the liquefact substrate for a period of time prior to inoculation with yeast. During this pre-incubation, or “seed period,” the fungal strains are able to utilize the small amounts of glucose present in the starting liquefact, e.g., 0.70 to 1.0% w/v, to initiate growth and begin protein expression. To seed the liquefact, 100 grams of whole liquefact at pH 4.8 containing 600 ppm urea were inoculated with a 4.5 ml glycerol stock of either A. niger or T. reesei. These seed flasks were incubated at 32° C. with mixing at 200 rpm for 9 hours. TABLE 1 describes the conditions tested, including exogenous GA, AA, and Acid Fungal Protease (AFP) doses.

At SSF time=0, 0.1% w/v Ethanol Red® active dry yeast (ADY) were added to all fermentation flasks. For flasks containing the A. niger strain, no exogenous GA or AA were added at SSF time=0, because A. niger expresses both the glucoamylase and alpha-amylase required for SSF. For flasks containing the T. reesei strain, exogenous GC626 and FERMGEN™ 2.5× were added at SSF time=0 at a 0.0053% w/w dose and a 0.00029% w/w dose, respectively. Positive controls with 0.054% w/w TrGA, 0.0053% w/w GC626, and 0.00029% w/w FERMGEN™ 2.5× were run under conventional SSF conditions. All fermentation conditions were tested at three temperatures: 32° C., 35° C., and a high temperature, 38° C. staged condition. Negative controls, containing only yeast and no exogenous enzymes, were also run at each temperature. Under the 38° C. staged temperature condition an additional negative control was run containing only yeast, 0.0053% w/w GC626, and 0.00029% w/w FERMGEN™ 2.5×.

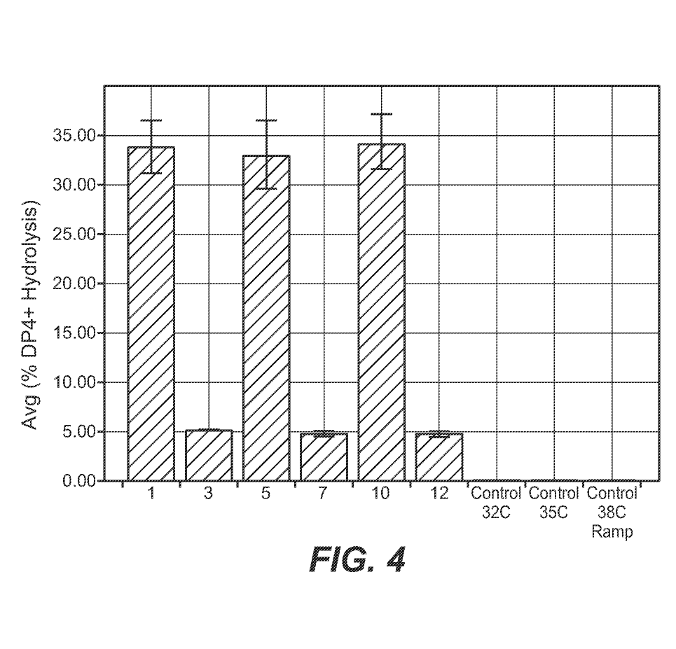

Following the seed incubation, high levels of glucose were liberated from liquefact containing the A. niger fungal strain, indicating significant levels of GA expression. After the 9 hour seed, flasks inoculated with the A. niger strain contain an average about 7-fold more glucose than in the starting liquefact. Also, since A. niger can co-express GA and AsAA, DP4+ yields are significantly reduced following the 9 hour seed incubation. At SSF time=0, flasks containing A. niger have 34% lower levels of DP4+ than in the starting liquefact. Because the AsAA works synergistically with the GA to hydrolyze DP4+, the reduction in DP4+ in flasks containing A. niger (blends 1, 5, and 10) is significantly higher than in flasks containing T. reesei (blends 3, 7, and 12), which can only express the GA. The synergistic effect of expressed A. niger enzymes, compared to T. reesei, is shown in FIG. 4 as a percent of total DP4+ hydrolysis.

Alternatively, a fungal inoculation was performed by pitching both the yeast and A. niger or T. reesei fungal strains directly into whole ground corn liquefact at SSF time=0 under the 38° C. staged temperature condition. As stated above, A. niger can express its own GA and AsAA and does not require the addition of exogenous enzymes at SSF time=0. For flasks containing the T. reesei strain, exogenous GC626 and FERMGEN™ 2.5× were added at SSF time=0 at a 0.0053% w/w dose and a 0.00029% w/w dose, respectively. Positive controls with 0.054% w/w TrGA, 0.0053% w/w GC626, and 0.00029% w/w FERMGEN™ 2.5× were run under conventional SSF conditions. Negative controls containing no exogenous enzyme or GC626 and FERMGEN™ 2.5× only were also run. TABLE 1 describes the conditions tested.

Specific results obtained with various, representative blends disclosed herein are discussed in more detail below.

Blend 1: A. niger Seed+Ethanol Red® Active Dry Yeast (ADY) Pitch at 32° C.

Under the typical operating temperature, 32° C., blend 1 containing only the A. niger fungal strain and Ethanol Red® yeast produces 86% of the total ethanol yield observed from the control blend and 4.6-fold more ethanol than the negative control (TABLE 2). 94% of the starting DP4+ concentration is hydrolyzed by the end of fermentation using blend 1 indicating significant levels of both GA and AA are produced during fermentation (TABLE 2).

Blend 5: A. niger Seed+Ethanol Red® ADY Pitch at 35° C.

As fermentation temperatures climb above 32° C., the yeast undergo thermal stress.

As a result the control blend at 35° C. yields 4% less ethanol than the control blend at 32° C. (96% versus 100%) (TABLE 2).

Blend 5 containing the A. niger fungal strain produces much more ethanol at 35° C. than at 32° C. (blend 1) (92% versus 86%), indicating increased enzyme expression and activity at higher temperatures (see TABLE 2). The opposite effect is seen using T. reesei (36% versus 53%) (see TABLE 2). At 35° C., blend 5 produces 92% ethanol compared to 96% ethanol produced by the control blend (see TABLE 2). These results indicate that A. niger is better able to withstand thermal stress than T. reesei and unexpectedly can be used in a co-culture with yeast to produce comparable yields of ethanol compared to a reaction supplemented with the addition of exogenous GA, GC626, and FERMGEN™ 2.5× Dose. DP4+ hydrolysis is slightly improved for blend 5 at 35° C., because the final DP4+ yield is 33% lower than blend 1 at 32° C. (TABLE 2). Blend 5 hydrolyzes 96% of the total DP4+, again indicating significant enzyme production during fermentation (TABLE 2).

Blend 10: A. niger Seed+Ethanol Red® ADY Pitch at 32-38-32° C.

Blend 10 contained only the A. niger fungal strain and Ethanol Red® yeast and was fermented using a staged temperature condition that ramps in a controlled fashion between 32° C. and 38° C. (see FIG. 3). The control for this reaction, Control 38° C. Ramp, contained Ethanol Red® yeast without an added fungal strain, and it contained exogenously added GA, GC626, and FERMGEN™ 2.5× Dose (see TABLE 1). Surprisingly, blend 10 produced significantly more ethanol than the control, 99% compared to 90%. (see TABLE 2). DP4+ hydrolysis for blend 10 was comparable to the control. These results show that A. niger not only can express all the enzymes needed for efficient ethanol production when co-cultured with yeast, but the A. niger can do so while under thermal stress.

Blend 13: A. niger+Ethanol Red® ADY Pitch at 32-38-32° C.

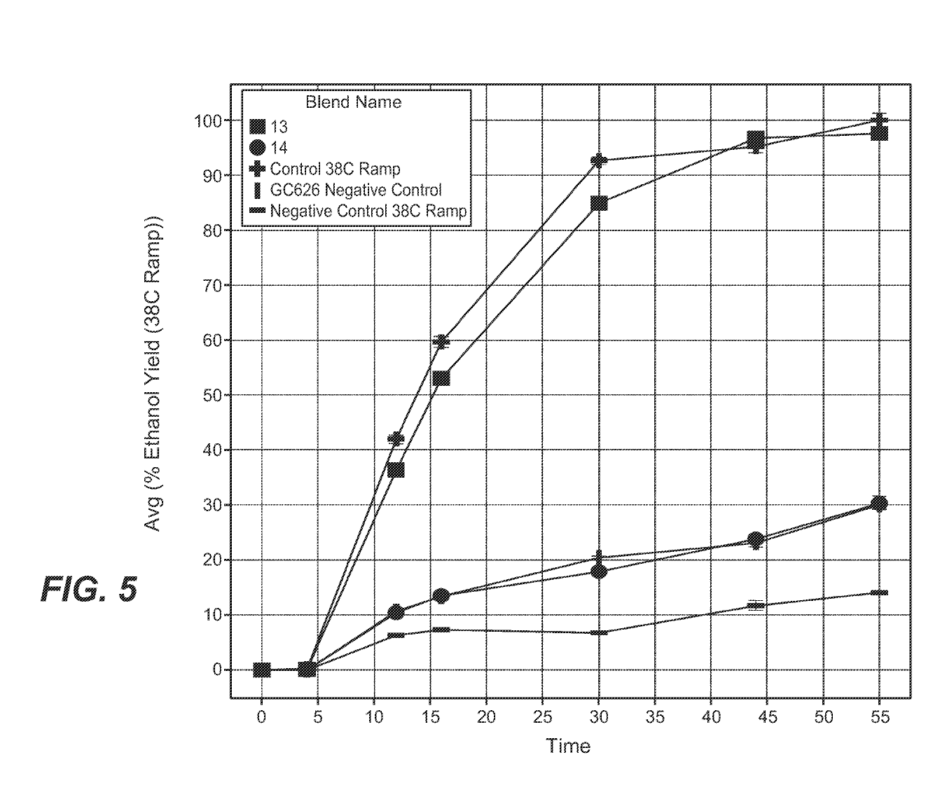

As with blend 10, both the Ethanol Red® yeast and A. niger fungal strain were inoculated, or pitched, into whole ground corn at SSF time=0. Using a staged temperature condition that ramps in a controlled fashion between 32° C. and 38° C. (FIG. 3), blend 13 containing only the pitched A. niger and Ethanol Red® yeast surprisingly produced 98% of the total ethanol yield compared to 90% for the control blend at the same temperature condition (TABLE 2, FIG. 5).

DP4+ hydrolysis for blend 13 is also comparable to levels observed with the control blend. Blend 13 hydrolyzes 96% of the total DP4+ at the staged temperature condition and is only 1.09-fold higher than the control blend at this temperature (TABLE 2).

Blends 3, 7, 12, and 14: T. reesei+Ethanol Red® ADY

Ethanol production is reduced in blends 3, 7, 12 and 14, compared to blends containing A. niger and the control blends (TABLE 2). On average, blends containing T. reesei produce 2.3-fold more ethanol than the negative control blends (36% to 66% of the control blend) at all temperature conditions compared to A. niger strains, which produce, on average, 5.2-fold more ethanol than the negative controls (86% to 99% of the control blend) (TABLE 2). This increase in ethanol production may be due to the exogenous addition of GC626 and not from the production of glucoamylase from T. reesei. DP4+ hydrolysis of only 73% of the total DP4+ may also be impacted by T. reesei's low glucoamylase production. Blends 3, 7, and 12 all have DP4+ levels averaging 9.9-fold higher than the control blends at end of fermentation (TABLE 2).

1. A conversion process of converting a starch substrate into an alcohol, comprising:
contacting a starch substrate with a yeast first cell and an Aspergillus niger second cell, wherein said conversion process produces an alcohol yield of at least 90% over a temperature range of about 32° C. to about 38° C. at the completion of the conversion process, compared to the alcohol yield at the completion of a control process performed under comparable conditions,
wherein the control process comprises contacting the starch substrate with the yeast first cell and adding an exogenous glucoamylase, fungal alpha-amylase, and fungal protease, and
wherein the conversion process produces the alcohol.

2. The conversion process of claim 1, wherein the alcohol is ethanol or butanol.

3. The conversion process of claim 1, wherein the conversion process is capable of producing an alcohol yield of at least 95-99% at the completion of the conversion process, compared to the alcohol yield at the completion of the control process.

4. The conversion process of claim 1, wherein the conversion process is performed over a temperature range of about 32° C. to about 38° C., and wherein the conversion process produces an alcohol yield of at least 90% at the completion of the conversion process, compared to the alcohol yield of the control process.

5. The conversion process of claim 1, wherein the conversion process is performed over a temperature range of about 35° C. to about 38° C., and wherein the conversion process produces an alcohol yield of at least 90% at the completion of the conversion process, compared to the alcohol yield of the control process.

6. The conversion process of claim 1, wherein the conversion process is performed at a temperature of about 35° C., and wherein the conversion process produces an alcohol yield of at least 90% at the completion of the conversion process, compared to the alcohol yield of the control process.

7. The conversion process of claim 1, further comprising pre-incubating the second cell with the starch substrate before the second cell and the starch substrate are contacted with the first cell.

8. The conversion process of claim 7, wherein said pre-incubating is conducted for 6-12 hours.

9. The conversion process of claim 1, wherein the starch substrate is a liquefact or granular starch.

10. The conversion process of claim 1, wherein the conversion process is conducted without addition of an exogenous glucoamylase, non-starch hydrolyzing enzyme, alpha-amylase, phytase, and/or protease.

11. The conversion process of claim 1, wherein the yeast first cell expresses an exogenous and/or endogenous glucoamylase, non-starch hydrolyzing enzyme, alpha-amylase, phytase, and/or protease during the conversion process.

12. The conversion process of claim 1, wherein the yeast first cell expresses an endogenous glucoamylase, non-starch hydrolyzing enzyme, alpha-amylase, phytase, and/or protease during the conversion process.

13. The conversion process of claim 1, wherein the yeast first cell expresses an exogenous glucoamylase, non-starch hydrolyzing enzyme, alpha-amylase, phytase, and/or protease during the conversion process.
